(19) United States
(12) Patent Application Publication
(10) Pub. No.: US 2021/0106712 A1
(43) Pub. Date: Apr. 15, 2021

(54) Charging Structure and aroma diffuser

(71) Applicant: Puzhen Life Co. Limited, Shatin (HK)

(72) Inventor: Andy LEE, Shatin (HK)

(21) Appl. No.: 16/728,467

(22) Filed: Dec. 27, 2019

(30) Foreign Application Priority Data
Oct. 12, 2019 (CN) 201921710421.8

(51) Int. Cl. A61L 9/03

(52) U.S. Cl. CPC A61L 9/03; A61L 2209/15

(57) ABSTRACT

A charging structure and an aroma diffuser suitable for a home furnishing small household electrical appliance. The appliance has a chassis and circuit, with a contact thimble arranged on the chassis and penetrating to a lower surface of the chassis. The contact thimble is connected to the circuit. An electrical connection component has a connector and a wire for connecting the connector. The wire is used for accessing a power source. The connector is electrically connected with the thimble when it is abutted with the contact thimble. A magnetic component has a first magnetic member disposed on the chassis and a second magnetic member disposed on the connector. The first magnetic member and second magnetic member are attracted to each other to abut the connector with the contact thimble. A horizontal projection of the chassis covers a horizontal projection of the connector.

### TECHNICAL FIELD

The present utility model belongs to the charging field of home furnishing small household electrical appliances, especially relates to a charging structure and an aroma diffuser.

### BACKGROUND ART

Small household appliances generally refer to household appliances other than high-power output electrical appliances. Generally, these small household appliances occupy relatively small power resources, or their body size is relatively small. Thus, they are called small household appliances. According to the functions of small household appliances, they can be divided into four categories: small kitchen household appliances, home furnishing small household electrical appliances, small personal living household appliances and personal digital products. Home furnishing small household electrical appliances include humidifiers, air fresheners, aromatherapy machines, etc. These devices are usually placed in the living room or in the room. In addition to their core functions (such as humidification, air purification and aromatherapy), consumers also care about their appearance when purchasing. The existing charging structure of a home furnishing small household electrical appliance is generally provided with a charging female connector on the home furnishing small household electrical appliance, and the home furnishing small household electrical appliance is connected to a socket or a plug through a wire with a male connector. For the convenience of plugging, a female connector is generally placed on the side surface of a home furnishing small household electrical appliance, thereby affecting the overall appearance.

### SUMMARY OF THE UTILITY MODEL

The present utility model is aimed to overcome the above-mentioned deficiencies of the prior art, and provides a charging structure and an aroma diffuser, which aims to solve the problem of ensuring the convenience of connection without affecting the home furnishing small household electrical appliances.

The present utility model is realized as follows:

A charging structure suitable for a home furnishing small household electrical appliance, wherein the household appliance comprises a chassis and a circuit, wherein the charging structure comprises:

a contact thimble arranged on the chassis and penetrating to a lower surface of the chassis, wherein the contact thimble is connected to the circuit;

an electrical connection component, comprising a connector and a wire for connecting the connector, wherein the wire is used for accessing a power source, and the connector is electrically connected with the thimble when it is abutted with the contact thimble;

a magnetic component, comprising a first magnetic member disposed on the chassis and a second magnetic member disposed on the connector, wherein the first magnetic member and the second magnetic member are attracted to each other to abut the connector with the contact thimble;

wherein a horizontal projection of the chassis covers a horizontal projection of the connector.

Further, the chassis is provided with an accommodating groove for accommodating the connector and an escaping groove for clamping the wire upwardly on a lower surface thereof, wherein when the connector is located in the accommodating groove and the wire is located in the escaping groove, the connector is abutted with the contact thimble.

Further, the escaping groove is connected to the wire by an interference fit.

Further, the contact thimble comprises a first thimble and a second thimble, the connector comprises a first guiding piece and a second guiding piece respectively for being abutted with the first thimble and the second thimble, the second guiding piece surrounds the first guiding piece and is spaced apart from the first guiding piece, and the wire comprises a first core connected with the first guiding piece and a second core connected with the second guiding piece.

Further, the connector further comprises a kit and a bottom frame connected from top to bottom, wherein the kit is provided with a first through hole and a second through hole penetrating up and down, the bottom frame is provided with a first blind hole and a second blind hole on an upper surface thereof, the bottom frame is provided with a connection hole communicating with the second blind hole on the side surface thereof, the first blind hole is used for accommodating the second magnetic member, the first core is connected is with the first guiding piece by passing through the connection hole, the second blind hole and the first through hole, and the second core is connected to the second guiding piece by passing through the connection hole, the second blind hole and the second through hole.

Further, the bottom frame and the kit are snap-fitted.

Further, the kit is provided with a first fixing groove for fixing a first guiding piece and a second fixing groove for fixing a second guiding piece downwardly on an upper surface thereof.

Further, the bottom frame and the kit are made of a rubber material.

Further, the connector is of a cylindrical shape extending upward and downward.

An aroma diffuser, comprising a charging structure as described above.

The charging structure provided by the present utility model attracts a connector to a lower surface of a chassis through the mutual attraction of a first magnetic member and a second magnetic member, and the connector is abutted with a contact thimble, which is very convenient to operate. In addition, the charging structure is located under the chassis so that the charging structure is prevented from protruding on the side position of the home furnishing small household electrical appliance, and the horizontal projection of the connector is covered by that of the chassis. Seen from a top view, the connector is hidden and seen from the side view the connector is also not easy to be found, thereby reducing the impact of the charging structure on the overall appearance of the home furnishing small household electrical appliance.

### BRIEF DESCRIPTION OF THE DRAWINGS

In order to more clearly illustrate the technical solutions of embodiments of the present utility model, the following contents focus on the drawings to be used for the embodiments. Apparently, the drawings in the following description are only some embodiments of the present utility model. Those skilled in the art may further obtain other drawings based on these drawings without any creative work.

FIG. 1 is a schematic diagram showing the connection of a charging structure and a chassis according to Embodiment 1 of the present utility model;

FIG. 2 is a schematic diagram of disassembly of a structure of FIG. 1;

FIG. 3 is a cross-sectional view of a structure of FIG. 1;

FIG. 4 is a schematic diagram of disassembly of a connector, a contact thimble and a second magnetic member in Embodiment 1 of the present utility model;

FIG. 5 is a cross-sectional view of a connector of Embodiment 1 of the present utility model.

### DESCRIPTION OF THE NUMBER IN THE FIGURE

| Number | Name |
|---|---|
| 10 | Chassis |
| 11 | Accommodating groove |
| 12 | Escaping groove |
| 20 | Contact thimble |
| 21 | First thimble |
| 22 | Second thimble |
| 30 | Electrical connection component |
| 31 | Connector |
| 311 | First guiding piece |
| 312 | Second guiding piece |
| 313 | Bottom frame |
| 3131 | First blind hole |
| 3132 | Second blind hole |
| 3133 | Connection hole |
| 314 | Kit |
| 3141 | First through hole |
| 3142 | Second through hole |
| 3143 | First fixing groove |
| 3144 | Second fixing groove |
| 32 | Wire |
| 40 | Magnetic component |
| 41 | First magnetic member |
| 42 | Second magnetic member |

### DETAILED DESCRIPTION

In order to make the objects, technical solutions and advantages of the present utility model clearer, the present utility model will be further described in detail below with reference to the attached drawings and embodiments. It should be understood that the specific embodiments described herein are merely illustrative of the present utility model and are not intended to limit the present utility model.

It needs to be noted that when an element is referred to as being “fixed” or “arranged” in another element, it may be directly or indirectly on the another element. When an element is referred to as being “connected” to another element, it may be directly or indirectly connected to the another element.

In addition, the terms “first” and “second” are used for descriptive purposes only and shall not be construed as indicating or implying relative importance or implicitly indicating the number of technical features indicated. Thus, features defining “first” and “second” may include one or more of the features explicitly or implicitly. In the description of the present utility model, “a plurality of” means two or more unless otherwise specifically defined.

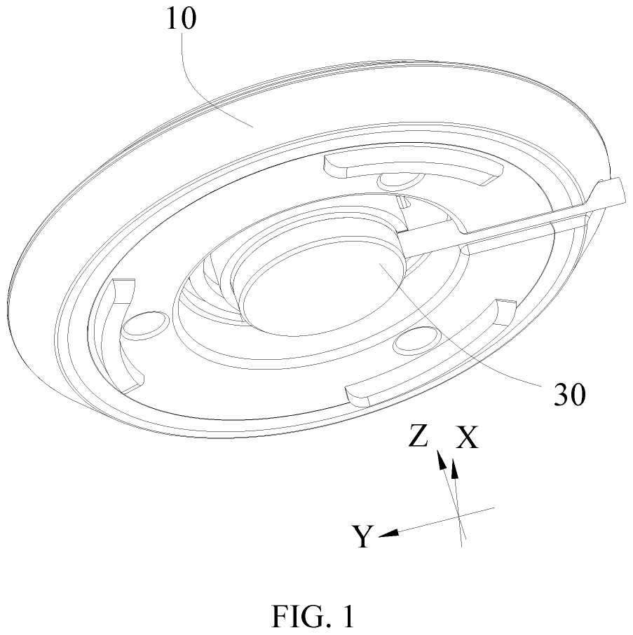

It should further be noted that, in the embodiments of the present utility model, according to the XYZ rectangular coordinate system established in FIG. 1, a side located in the positive direction of the X-axis is defined as the front side, and a side located in the negative direction of the X-axis is defined as the rear side; a side located in the positive direction of the Y-axis is defined as the left side, a side located in the negative direction of the Y-axis is defined as the right side; a side located in the positive direction of the Z-axis is defined as the upper side, and a side located in the negative direction of the Z-axis is defined as the lower side.

It should further be noted that the left, right, upper and lower orientation terms in the embodiments of the present utility model are only relative concepts or with reference to the normal use state of the product, and should not be considered as restrictive.

### Embodiment 1

Please refer to FIG. 1 to FIG. 5. The present embodiment provides a charging structure suitable for a home furnishing small household electrical appliance, and the home furnishing small household electrical appliance comprises a chassis 10 and a circuit (not shown in the figure). Those skilled in the art may understand that a home furnishing small household electrical appliance further comprises a household electrical appliance body (not shown in the figure), the chassis 10 is for supporting the household electrical appliance body and that the chassis 10 and the household electrical appliance body are integrated or fixedly connected. The circuit is arranged in the household electrical appliance body to realize the use function of the home furnishing small household electrical appliance together with the household electrical appliance body.

The charging structure comprises a contact thimble 20, an electrical connection component 30 and a magnetic component 40.

The contact thimble 20 is arranged on the chassis 10 and penetrating to a lower surface of the chassis 10, wherein the contact thimble 20 is connected to the circuit.

The electrical connection component 30 comprises a connector 31 and a wire 32 for connecting the connector 31, wherein the wire 32 is used for accessing a power source, and the connector 31 is electrically connected with the thimble when it is abutted with the contact thimble 20;

A magnetic component comprises a first magnetic member 41 disposed on a chassis 10 and a second magnetic member 42 disposed on a connector 31, wherein the first magnetic member 41 and the second magnetic member 42 are attracted to each other to abut the connector 31 with a contact thimble 20;

wherein, a horizontal projection of the chassis 10 covers a horizontal projection of the connector 31.

When the charging structure provided by the present embodiment is used, the connector 31 is close to a lower surface of the chassis 10 so as to attract the connector 31 to the lower surface of the chassis 10 through the mutual attraction of the first magnetic member 41 and the second magnetic member 42, and the connector 31 is abutted with the contact thimble 20 so as to realize electrical connection between the connector 31 and the contact thimble 20. The operation is very convenient.

In addition, the charging structure is located under the chassis 10 so that the charging structure is prevented from protruding on the side position of the home furnishing small household electrical appliance, and the horizontal projection of the connector 31 is covered by that of the chassis 10. Seen from a top view, the connector 31 is hidden and seen from the side view the connector 31 is also not easy to be found, thereby reducing the impact of the charging structure on the overall appearance of the home furnishing small household electrical appliance.

It should be further noted that in terms of the connection between the existing male connector 31 and the female connector 31, in order to ensure the firm connection between the male connector 31 and the female connector 31, the male connector 31 and the female connector 31 are usually connected in a nested manner. The male connector 31 is large in size. If the connection of the male connector 31 and the female connector 31 is directly transferred to a lower side of the chassis 10, it occupies large vertical space. The charging structure provided by the present solution may be electrically connected by abutting the contact thimble 20 with the connector 31, and the chassis 10 and the connector 31 are connected by the magnetic component 40, so that the size of the connector 31 may be flat and miniaturized, thereby effectively reducing the vertical space occupied, further conducive to its own hiding as well as reducing is the impact on the overall appearance of the home furnishing small household electrical appliance.

It should be further explained that the present solution is different from a charging structure of a kettle. A kettle comprises a kettle body, a tray and a charging structure. The charging structure of the kettle comprises a connector disposed on the kettle body, a metal protrusion disposed on the tray and a wire for connecting the metal protrusion. The kettle body and the tray are provided with a corresponding card position respectively. When the kettle body is clamped onto the tray, the connector is abutted with the metal protrusion to achieve electrical connection between the kettle body and the wire. In this connection, the kettle body is connected to the tray by the weight of the kettle body. The present solution adopts a magnetic connection, which is different from this solution and is not a simple replacement. Specifically, if the chassis 10 is pressed against the connector 31 by the weight of the main body of the household appliance, since the main body of the household appliance and the chassis 10 are not perspective, in order to achieve the aligned clamping of the chassis 10 and the connector 31, the size of the connector 31 is required to be set to be equal to or larger than that of the chassis 10, which is contrary to the purpose of hiding the connector 31 as much as possible in the present solution. In the present solution, the alignment is realized mainly through mutual attraction of the first magnetic member 41 and the second magnetic member 42. Thus, the charging structure of the present solution is different from a charging structure of a kettle and is not a simple replacement.

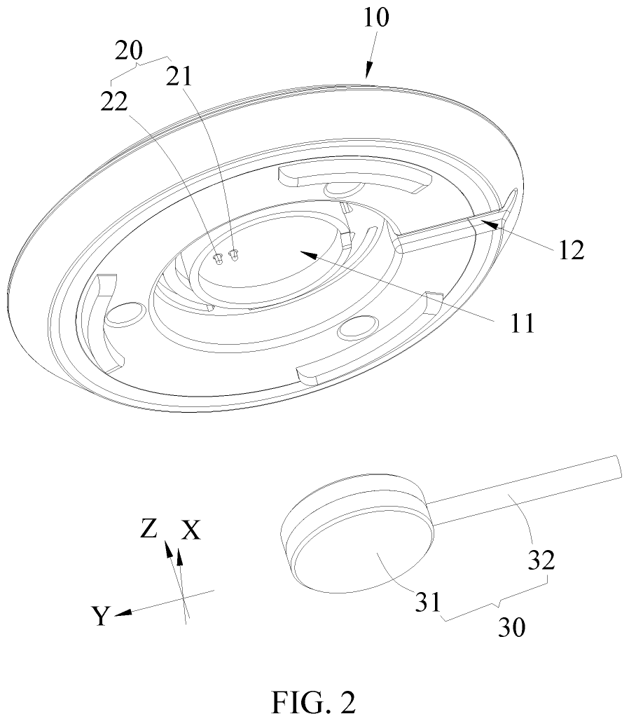

Please refer to FIG. 2. A wire 32 extends in the horizontal direction and one end thereof is connected to the side surface of a connector 31. Such design is advantageous for reducing the vertical dimension of an electrical connection component 30, thereby facilitating the hiding of an electrical connection component 30.

Please refer to FIG. 2. A chassis 10 is provided with an accommodating groove 11 for accommodating a connector 31 and an escaping groove 12 for escaping a wire 32. When the connector 31 is located in the accommodating groove 11 and the wire 32 is located in the escaping groove 12, the connector 31 is abutted with a contact thimble 20. The arrangement of the accommodating groove 11 defines the movement of the connector 31 in the horizontal plane to further ensure accurate alignment of the connector 31 and the chassis 10. Furthermore, the arrangement of the accommodating groove 11 enables the connector 31 to be embedded in the chassis 10, thereby further hiding the connector 31.

Preferably, the depth of the accommodating groove 11 is comparable to the height of the connector 31 so that the connector 31 is completely hidden in a bottom frame 313. In the illustrated embodiment, the depth of the accommodating groove 11 is less than the height of the connector 31, and three supporting protrusions are provided under the chassis 10 to lift the chassis 10 and the connector 31 off the ground. Since the size of the connector 31 protruding from the chassis 10 is smaller than the horizontal size of the chassis 10, the effect of hiding the connector 31 can also be satisfied in normal use.

The escaping groove 12 is for accommodating a wire 32. Considering that the wire 32 is generally horizontally extended and connected to the side of the connector 31, the escaping groove 12 can accommodate the wire 32, so that the connector 31 can smoothly enter the accommodating groove 11.

Preferably, an interference fit between the escaping groove 12 and the wire 32 is achieved. The wire 32 is located in the escaping groove 12 and is pressed by the escaping groove 12 so as to fix the wire 32. In consideration of the connection relationship of the connector 31 and the wire 32, the design can further improve the firm connection between the electrical connection component 30 and the chassis 10 so as to improve the firm connection between the connector 31 and the contact thimble 20.

In the present embodiment, the connector 31 is of a cylindrical shape is extending upward and downward, and the accommodating groove 11 is adapted to the connector 31. In other embodiments, the connector 31 may also be a prism extending upward or downward, or a cylinder, a hemisphere or the like. The accommodating groove 11 is adapted to the shape of the connector 31 and is not limited thereto.

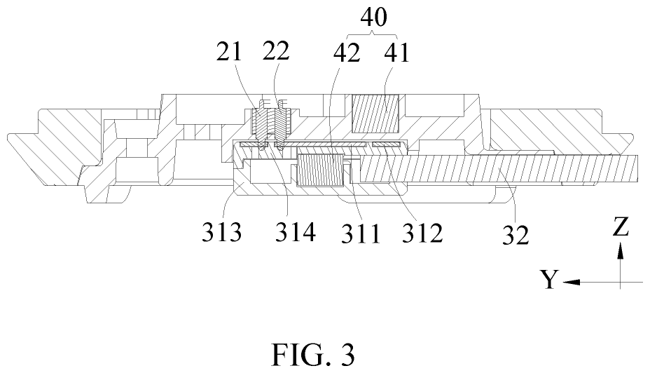

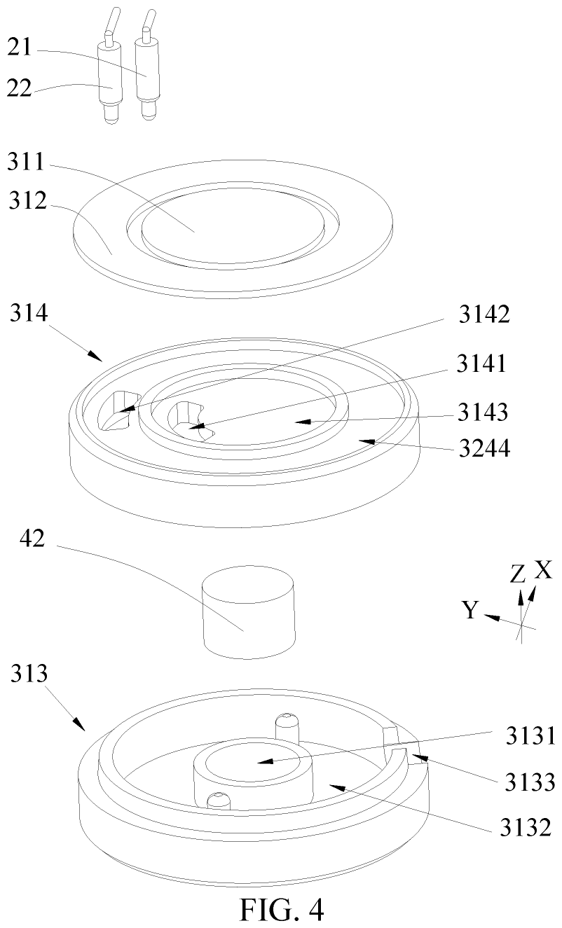

Please refer to FIG. 3 and FIG. 4. The contact thimble 20 comprises a first thimble 21 and a second thimble 22, the connector 31 comprises a first guiding piece 311 and a second guiding piece 312 respectively for being abutted with the first thimble 21 and the second thimble 22, the second guiding piece 312 surrounds the first guiding piece 311 and is spaced apart from the first guiding piece 311, and the wire 32 comprises a first core connected with the first guiding piece 311 and a second core connected with the second guiding piece 312. Those skilled in the art may understand that the first core is a live wire, the second core is a neutral wire, or conversely, the first core is a neutral wire and the second core is a live wire. The first guiding piece 311 and the second guiding piece 312 are spaced apart to avoid short circuiting. The second guiding piece 312 is annular and is disposed outside the first guiding piece 311, so that the second guiding piece 312 can be connected with the second thimble 22 and the first guiding piece 311 can be connected to the first thimble 21 regardless of whether the connector 31 rotates at an arbitrary angle around the center of the second guiding piece 312, thereby making operation more convenient. Especially in the case where the accommodating groove 11 and the escaping groove 12 are not provided, the design greatly reduces the alignment requirement, thereby making operation more convenient. In the illustrated embodiment, the first guiding piece 311 is a circular plate. In other embodiments, the first guiding piece 311 may also be an annular plate.

In combination with the foregoing contents, the connector 31 is of a cylindrical shape extending upward and downward, the first guiding piece 311 is a circular plate, and the second guiding piece 312 is an annular plate. In the is design, the connector 31 and the contact thimble 20 can be abutted just by accommodating the connector 31 into the accommodating groove 11, without paying attention to the horizontal placement angle of the connector 31, thereby further reducing the alignment requirement.

It should be noted that the sheet-like arrangement of the first guiding piece 311 and the second guiding piece 312 further helps to reduce the vertical dimension of the electrical connection component 30, thereby facilitating the hiding of the electrical connection component 30.

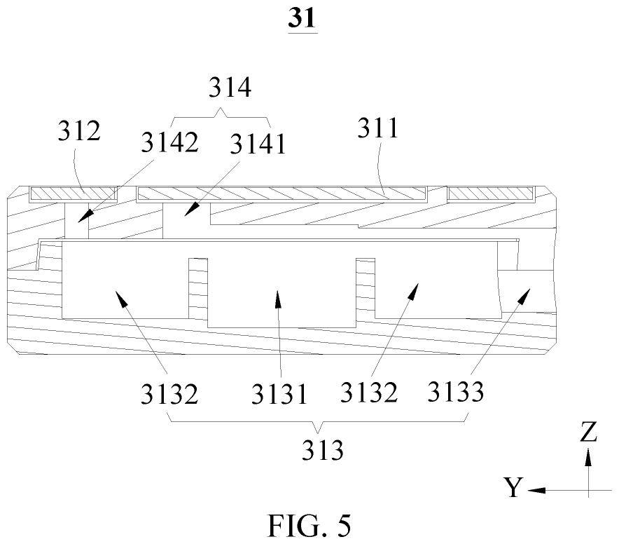

Please also refer to FIG. 5. The connector 31 further comprises a kit 314 and a bottom frame 313 connected from top to bottom, wherein the kit 314 is provided with a first through hole 3141 and a second through hole 3142 penetrating up and down, the bottom frame 313 is provided with a first blind hole 3131 and a second blind hole 3132 on an upper surface thereof, the bottom frame 313 is provided with a connection hole 3133 communicating with the second blind hole 3132 on the side surface thereof, the first blind hole 3131 is used for accommodating the second magnetic member 42, the first core is connected with the first guiding piece 311 by passing through the connection hole 3133, the second blind hole 3132 and the first through hole 3141, and the second core is connected to the second guiding piece 312 by passing through the connection hole 3133, the second blind hole 3132 and the second through hole 3142.

The bottom frame 313 and the kit 314 are separately provided, which not only facilitates the processing thereof but also facilitates the placement operation of the second magnetic member 42 and the assembly operation of the wire. The specific assembly process is as follows. The second magnetic member 42 is placed in the first blind hole 3131, the wire 32 is extended from the side of the bottom frame 313 into the connection hole 3133, the first core passes through the second blind hole 3132 and the first though hole 3141, the second core passes through the second blind hole 3132 and the second through hole 3142, and then the kit 314 is snap-fitted to the bottom frame 313. At this time, the first core and the second core protrude from the kit 314. Then, the first guiding piece 311 and the second guiding piece 312 are pressed against the kit 314 and are respectively connected to the first core and the second core.

The bottom frame 313 and the kit 314 are snap-fitted to achieve mutual alignment, and the bottom frame 313 and the kit 314 may be connected by ultrasonic connection or threaded connection, etc., which is not limited herein.

Please refer to FIG. 4. The kit 314 is provided with a first fixing groove 3143 for fixing a first guiding piece 311 and a second fixing groove 3144 for fixing a second guiding piece 312 downwardly on an upper surface thereof The arrangement of the first fixing groove 3143 and the second fixing groove 3144 limits the horizontal movement of the first guiding piece 311 and the second guiding piece 312, and shields the abutting surface between the lower surface of the first guiding piece 311 and the second guiding piece 312 and the kit 314, thereby preventing the external force from acting on the abutting surface so as to separate the first guiding piece 311 and the second guiding piece 312 from the kit 314. Preferably, the depth of the first fixing groove 3143 is equivalent to the thickness of the first guiding piece 311, and the depth of the second fixing groove 3144 is equivalent to the thickness of the second guiding piece 312, thereby shielding the side surface of the first guiding piece 311 and the second guiding piece 312 so as to prevent the external force from directly acting on the side surface of the first guiding piece 311 and the second guiding piece 312 to easily separate the first guiding piece 311 and the second guiding piece 312 from the kit 314 to further improve the firm connection between the first guiding piece 311 and the second guiding piece 312 and the kit 314.

In the present embodiment, the bottom frame 313 and the kit 314 are made of an insulating material, preferably a rubber material. The rubber material has sufficient hardness to make and fix the first guiding piece 311, the second guiding piece 312 and the second magnetic member 42, and the material cost is is low.

In the present embodiment, the first magnetic member 41 is made of a magnet material, and the second magnetic member 42 is made of a magnet material or an iron metal material. Those skilled in the art may select the second magnetic member 42 made of a magnet material and the first magnetic member 41 made of an iron metal material, as long as the first magnetic member 41 and the second magnetic member 42 are capable of generating mutually opposing forces when they are close to each other so that the electrical connection component 30 can be attracted to the lower surface of the chassis 10.

### Embodiment 2

The present embodiment provides an aroma diffuser for evacuating a liquefied essential oil to the outside, and the aroma diffuser comprises a charging structure. The specific structure of the charging structure is referred to the foregoing Embodiment 1. Since all the technical solutions of the above-mentioned Embodiment 1 are adopted in the present aroma diffuser, all the beneficial effects of the technical solutions of the above-mentioned embodiment can be achieved and are not described herein.

The above-described embodiments are only preferred ones of the present utility model and do not limit the present utility model. Any modification, equivalent replacement or improvement based on the spirit and principles of the present utility model should be included in the protection scope of the present utility model.

1. A charging structure suitable for a home furnishing small household electrical appliance, wherein the home furnishing small household electrical appliance comprises a chassis and a circuit, wherein the charging structure comprises:
a contact thimble arranged on the chassis and penetrating to a lower surface of the chassis, wherein the contact thimble is connected to the circuit;
an electrical connection component, comprising a connector and a wire for connecting the connector, wherein the wire is used for accessing a power source, and the connector is electrically connected with the thimble when it is abutted with the contact thimble;
a magnetic component, comprising a first magnetic member disposed on the chassis and a second magnetic member disposed on the connector, wherein the first magnetic member and the second magnetic member are attracted to each other to abut the connector with the contact thimble;
wherein a horizontal projection of the chassis covers a horizontal projection of the connector.

2. The charging structure according to claim 1, wherein the chassis is provided with an accommodating groove for accommodating the connector and an escaping groove for clamping the wire upwardly on a lower surface thereof, wherein when the connector is located in the accommodating groove and the wire is located in the escaping groove, the connector is abutted with the contact thimble.

3. The charging structure according to claim 2, wherein the escaping groove is connected to the wire by an interference fit.

4. The charging structure according to claim 1, wherein the contact thimble comprises a first thimble and a second thimble, the connector comprises a first guiding piece and a second guiding piece respectively for being abutted with the first thimble and the second thimble, the second guiding piece surrounds the first guiding piece and is spaced apart from the first guiding piece, and the wire comprises a first core connected with the first guiding piece and a second core connected with the second guiding piece.

5. The charging structure according to claim 4, wherein the connector further comprises a kit and a bottom frame connected from top to bottom, wherein the kit is provided with a first through hole and a second through hole penetrating up and down, the bottom frame is provided with a first blind hole and a second blind hole on an upper surface thereof, the bottom frame is provided with a connection hole communicating with the second blind hole on the side surface thereof, the first blind hole is used for accommodating the second magnetic member, the first core is connected with the first guiding piece by passing through the connection hole, the second blind hole and the first through hole, and the second core is connected to the second guiding piece by passing through the connection hole, the second blind hole and the second through hole.

6. The charging structure according to claim 5, wherein the bottom frame and the kit are snap-fitted.

7. The charging structure according to claim 5, wherein the kit is provided with a first fixing groove for fixing a first guiding piece and a second fixing groove for fixing a second guiding piece downwardly on an upper surface thereof.

8. The charging structure according to claim 5, wherein the bottom frame and the kit are made of a rubber material.

9. The charging structure according to claim 1, wherein the connector is of a cylindrical shape extending upward and downward.

10. An aroma diffuser, comprising a charging structure according to claim 1.
